(19) United States
(12) Patent Application Publication
(10) Pub. No.: US 2021/0390508 A1
(43) Pub. Date: Dec. 16, 2021

(54) System For Controlling The Spread Of Infectious Diseases

(71) Applicant: Bipin KOLLURI, Fairview, TX (US)

(72) Inventor: Bipin KOLLURI, Fairview, TX (US)

(21) Appl. No.: 17/345,711

(22) Filed: Jun. 11, 2021

Related U.S. Application Data
(60) Provisional application No. 63/038,005, filed on Jun. 11, 2020.

(51) Int. Cl. G06Q 10/10; G16H 10/40

(52) U.S. Cl. CPC G06Q 10/105; A61B 5/6887; G16H 10/40

(57) ABSTRACT

This invention relates to systems and methods for controlling the spread of microorganism infections or infectious disease. Present invention is particularly directed to providing systems and methods that allow secure distribution of self-expiring health badges to the subjects free of Severe acute respiratory syndrome coronavirus 2 (SARS-CoV-2) infection. Individuals desirious of reducing the risks of infection and preventing the spread of such diseases participate in the testing program whereby regular periodic screening tests are performed to indicate the infection status of each such individual. The self-expiring badge given to infection-free individuals uses a time indicator and provides a clear indication of expiration of the badge after a predetermined period of time interval. Furthermore, the individuals may be employees of a business entity and testing all employees periodically can provide an infection-free work environment.

### CROSS-REFERENCE TO RELATED APPLICATIONS

This non-provisional application claims benefit of provisional application U.S. Ser. No. 63/038,005 filed Jun. 11, 2020, the entirety of which is hereby incorporated by reference.

### BACKGROUND OF THE INVENTION

### Field of the Invention

The present invention generally relates to the fields of medicine and molecular biology and particularly related to screening methods and systems for microorganism infections. Present invention is more particularly related to methods for preventing the spread of infectious diseases which are transmissable among humans through contact and to methods for assessing the health status of a person and secure distribution of health badges to the infection-free subjects.

### SUMMARY OF THE INVENTION

It is an object of the present invention to provide a method of preventing the spread of infections, principally through behavior modification.

The present invention is directed to a method for controlling the spread of microorganism infections or infectious disease. The method comprising the steps of: (a) identifying a group of persons desirous of controlling the spread of microorganism infections or infectious disease; (b) periodically testing said persons in said group to obtain infection status data for each person; and (c) issuing a self-expiring identification badge upon said person in said group tests negative for said microorganism infections or infectious disease.

The present invention is further directed to a method for controlling the spread of microorganism infections or infectious disease. The method comprising the steps of: (a) identifying a group of persons desirous of controlling the spread of microorganism infections or infectious disease; (b) periodically testing said persons in said group to obtain infection status data for each person; and (c) issuing a self-expiring identification badge upon said person in said group tests negative for said microorganism infections or infectious disease; where said infection or infectious disease is caused by Severe Acute Respiratory Syndrome Coronavirus 2 (SARS-CoV-2).

The present invention is directed further still to a method for controlling the spread of Severe Acute Respiratory Syndrome Coronavirus 2 (SARS-CoV-2) infection and corona virus disease. The method comprising the steps of: (a) identifying a group of persons desirous of controlling the spread of SARS-CoV-2 infection and corona virus disease; (b) collecting a blood sample from the person in said group; (c) testing the blood sample for SARS-CoV-2 infection and producing a result; and (d) issuing a self-expiring identification badge upon said person in said group tests negative for said SARS-CoV-2 infection and corona virus disease.

While various aspects of the present invention have been particularly shown and described with reference to the exemplary, non-limiting, embodiments above, it will be understood by those skilled in the art that various additional aspects and embodiments may be contemplated without departing from the sprit and scope of the present invention,

### BRIEF DESCRIPTION OF THE DRAWINGS

FIG. 1 is a block diagram of the system architecture of the platform of the present invention.

FIG. 2 is the block diagram of a kiosk of the present invention.

FIG. 3 is a flow chart illustrating the algorithm implemented by the kiosk software.

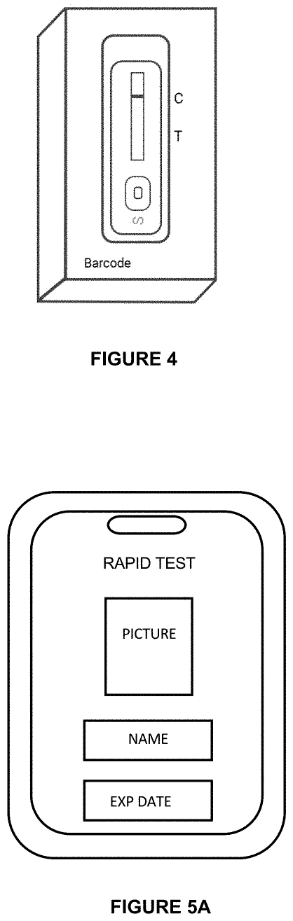

FIG. 4 is a rapid-test cassette used in the present invention.

FIGS. 5A-5B are front view of an exemplary self-expiring badge/card.

FIGS. 6-7 is an exemplary kiosk of the present invention.

FIG. 8 is a flow diagram for testing and issuance of self-expiring badge at the Business location.

FIG. 9 is a flow diagram for issue of Certificate of Safety.

FIG. 10 is a representational Certificate of Safety of the present invention.

### DETAILED DESCRIPTION OF THE INVENTION

As used herein the specification, “a” or “an” may mean one or more. As used herein, the terms “subject”, “customer”, “person” and “user” frequently refer to a human subject. Throughout the invention, the terms subject, customer, person and user are used interchangeably. As used herein the term “sample” refers to anything which may contain an analyte for which an analyte assay is desired. The sample may be a biological sample, such as a biological fluid. Examples of biological fluids include blood, plasma, serum, saliva, mucus, tears, and semen. Other biological samples (e.g., urine, stool) can also be contemplated. As used herein, the term ‘antigen’ refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein, microorganisms can include any of the viruses, bacteria, yeast, or parasites.

The viruses can include but not limited to Adenovirus, Adenovirus F40/41, Astrovirus, Cororiavirus, Coronavirus 229E, Coronavirus, Coronavirus NL63, Coronavirus 0C43, Cytomegalovirus (CMV), Enterovirus, Herpes simplex virus I (HSV-1), Herpes simplex virus 2 (HSV-2), Human herpesvirus 6 (HHV-6), Human Metapneumonvirus, Human Metapneumovirus, Human parechovirus, Human Rhinovirus/Enterovirus, Influenza A. Influenza A/H1, Influenza NH1-2009, Influenza NH3, Influenza B, Middle East Respiratory Syndrome Coronavirus, Norovirus GI/GII, Parainfluenza virus, Parainfluenza Virus 1, Parainfluenza Virus 2, Parainfluenza Virus 3, Parainfluenza Virus 4. Respiratory Syncptial Virus, Respiratory Syncytial Virus, Rotavirus A, Sapovirus (I, II, IV and V), Severe acute respiratory syndrome coronavirus 2 (SARS-CoV-2), and Varicella zoster virus (VZU).

The bacteria can include but not limited to E. coli 0157, Chlamydia pneurnortiae, E. coli (STEC) stxilstx2, Enteroaggregative E. coli (EAEC), Enterobacter cloacae complex, Enteropathogenic E. coli (EPEC), Enterotoxigenic E. coli (ETEC) It/st, Escherichia coli, Klebsiella oxytoca, Klebsiella pneumoniae, Legionella pneumophila, Mycoplasma pneumoniae, Proteus, Shiga-like toxin-producing, Shigella/Enteroinvasive E. coli (EIEC), Streptococcus pneumoniae, Streptococcus pyogenes, Acinetobacter baumannii, Acinetobacter calcoaceticus-baumannii complex, Antimicrobial Resistance Genes CTX-M, Antimicrobial Resistance Genes IMP, Antimicrobial Resistance Genes ITC, Antimicrobial Resistance Genes mecA/C and MREJ, Antimicrobial Resistance Genes kIDM, Antimicrobial Resistance Genes Oxa48-like, Antimicrobial Resistance Genes VIM, Atypical Bateria:, Bordetella parapertussis, Bordetella pertussis, Campylobacter (jejuni, coli and upsaliensis), Chlamydia (Chlamyclophila) pneumoniae, Clostridium (toxin NB), Diarrheagenic E. coli/Shigella:, Enerobacteriaceae:, Enterobacter aerogenes, Enterococcus, Entrogacter cloacae, Escherichia coli, Escherichia coli KI Haemophilus influenza, Haemophilus influenzae, Heamophilus influenza, Klebsiella oxytoca, Klebsiella pneumoniae group, KPC-carbapenem resistance, Listeria monocytogenes, mec.A-methicillin resistance, Morazella catarrhalis, Mycoplasma pneumonia, Neisseria meningitidis, Neisseria meningtidis, Plesiomonas shigeliodies, Proteus spp., Pseudomonas aeruginosa, Quantitative Bacteria, Salmonella, Semi-Quantitative Bacteria:, Serratia marcescens, Staphylococcus aureus, Steptococcus agalactiae, Stetococcus pneumoniae, Streptococccus phenummiae, Streptococcus agalactiae, Streptococcus pyogenes, vanA/B vancomycin resistance, Vibrio (parahaemolyticus, vulnificus and cholerae), Vibrio cholerae, Yersinia enterocolitica, Enterococcus-Listeria monocytogenes, Staphylococcus-Staphylococcus aureus, Streptococcus-Streptococcus agalactiae, Streptococcus pneurnoniae, Streptococcus pyogenes, Campylobacter (jejuni, coli and upsaliensis), Clostridium (toxin NB), Plesiomonas shigellodies, Salmonella, Yersinia enterocolitica, (parahaemolyticus, vulnificus and cholerae), Vibrio cholerae, Diarrheagenic E.coli/Shigella-Enteroaggregative E. coli (EAEC), Enteropathogenic E. coli (EPEC), Enterotoxigenic E. coli (ETEC) It/st, Shiga-like toxin-producing, E. coli (STEC) stx1/stx2, E, coli 0157, and Shigella/Enteroinvasive E. coli (EEC),

The yeast can include but not limited to Candida albicans, Candida glabrata, Candida krusei, Candida parapsilosis, Candida tropicalis, and Cryptococcus neoformansigatti.

The parasites can include but not limited to Cryptosporidium, Cyclospora cayetanensis, Entamoeba histolytica, and Giardia lamblia.

Exemplary, non-limiting embodiments of the present invention are discussed in detail below. While specific configurations, methods and drawings are discussed to provide clear understanding, it should be understood that the specific embodiments and theft respective configurations are provided for illustration purposes only. A person skilled in the relevant art will recognize that other configurations may be used without departing from the spirit and scope of the invention.

### General Embodiments of the Invention

Present invention is related to systems, methods and kits for assessing the health status of a person and providing self-expiring health badges to the infection-free subjects. The systems, methods and kits for health screening and secure distribution of self-expiring health badges can be used at any suitable location. In preferred embodiments, the systems, methods and kits for assessing subjects' exposure to SARS-CoV-2 and providing self-expiring health badges to the infection-free subjects can be used at a point of presence. Exemplary point of presence can be a retail location or a store, e.g., a pharmacy store or a supermarket, restaurants, malls, schools, airports, public places (e.g. parks), a medical office, a clinical laboratory, a health station, a gym, or any heavy traffic moving locations that are convenient and easily accessible.

The systems, methods and kits for controlling the microorganism infections or infectious disease can conduct a SARS-CoV-2 infection test and issue a self-expiring health badges to infection free subjects within any suitable time period, e.g. in about 1 hours, 50 minutes, 40 minutes, 30 minutes, 20 minutes, 10 minutes, or 5 minutes, from the time a sample is obtained from the subject.

The present systems, methods and kits for health screening can further comprise a means for assessing an additional health indicator of the subjects. Any suitable means can be used, In some embodiments the means is used to assess weight (body mass index), blood pressure, heart rate and oxygen saturation.

Exemplary advantages of a system in accordance with the present system include testing the employees of business entities and provided a health badge to the infection-free employees. Once all the employees are tested negative, the business entity may be provided with a “Certificate of Safety” which can be prominently displayed in the visible areas of business (e.g. restaurants). This helps in boosting customer confidence in the business entity and use the products and services of the business with peace of mind.

### User Registration

According to the present embodiment, the subject is requested to undergo the registration process. Any suitable means for proper identification and registration can be used. For example, typical registration process comprises verification of the subject's demographical information, e.g. name, address, phone number, employer information, valid photo identification card and sign the forms. Other methods such as a number of questionnaires can also be used.

In an exemplary process, the subjects can complete the registration and validation in multiple ways. The subject can complete the registration by downloading the fast-trac app on smart device (e.g., iOS, android, windows) or go to the online website using a web browser and complete the registration process. The subject may be prompted to upload a valid identification card and a photo (selfie) for validation, If the subjects are employees of a business entity, then the user registration, health screening and issuing self-expiring health badges can be arranged on-site at the business location upon the business entity sign up with the service provider.

The present invention also contemplates using a stand-alone self-service (self-help) registration system for completing the registration process. A self-serivce registration machine includes a computer system that includes a touch screen for receiving input information and completing the registration and validation process. The present system may further comprise a means for assessing an identifier of the subjects. Any suitable means can be used. In some embodiments the means is an iris scan, a fingerprinting device, a means for assessing voice identification, photograph, electronic signature, or photo identification. In all the embodiments and aspects of the present invention, the user data is stored securely on the cloud. The cloud service that provides such a service generally stores customer data remotely from the premises of the customer.

The present invention is not limited to any particular registration process. An ordinary skilled person is familiar with various registration methods and any suitable registration method can be used. Additionally, if the subjects are paying for the tests, the payments and financial transactions can be processed through the fast-trac app, website or manually at the business location. The users can pay with Fiat currency or crypto currency.

### Rapid Tests

The present disclosure provides kits for assessing subjects' exposure to SARS-CoV-2. The kit comprises rapid test devices, said rapid test devices are configured to assess analytes in a sample derived from the subjects. The analytes are selected from the group consisting of SARS-CoV-2 antigen, a SARS-CoV-2 polynucleotide, and an anti-SARS-CoV-2 antibody.

In one aspect, the present disclosure provides a test device for assessing subjects' exposure to SARS-CoV-2. The devices are configured to assess analytes in a sample derived from the subjects. The analytes are selected from the group consisting of SARS-CoV-2 antigen, a SARS-CoV-2 polynucleotide, and an anti-SARS-CoV-2 antibody.

In another aspect, the present disclosure provides a system for assessing subjects exposure to SARS-CoV-2, which system comprises any of the above kits and devices.

In yet another aspect, the present disclosure provides a method for assessing subjects' exposure to SARS-CoV-2. The devices are configured to assess analytes in a sample derived from the subjects. The analytes are selected from the group consisting of SARS-CoV-2 antigen, a SARS-CoV-2 polynucleotide, and an anti-SARS-CoV-2 antibody.

In some embodiments, the present system can further comprise a means for obtaining at least two different types of samples from the subjects. In other embodiments, the present system can further comprise a means for collecting multiple volumes of blood samples to run multiple assays. The device can make at least two punctures at once to increase the amount of blood sample volume needed to perform multiple assays on the blood sample.

In an embodiment, if the customer is tested positive for SARS-CoV-2 infection, the system provides instructions for the next course of action. For example, the customer can schedule an appointment with the physician for further examination and diagnosis (e.g. PCR testing). The positive results data may be shared with customer's physician or any assigned physician. All the customer data (excluding the private data) can be stored into Centers for Disease Control and Prevention (CDC) or National Contact Tracing Database.

### Respiratory Monitor

The devices and kits of present invention may include a respiratory monitor capable of pulmonary function testing in one embodiment, the respiratory monitor incorporates nitric oxide detectors where exhaled breadth from a customer enters the detection device and a NOₓ sensor measures the NOx concentration in the sample. Importantly, the sensors provide an accurate concentration of nitric oxide in the presence of other possibly interfering gas components (e.g. water, carbon dioxide). In one aspect, the respiratory monitor measures the concentration of fractionally exhaled nitric oxide (FeNO). In one aspect, the customer exhales into the device through a mouthpiece wherein the mouthpiece allows the customer to inhale outside air through the mounthpiece, and exhale through the mouthpiece and into the device through a path separate from the path of inhaled air. In a related aspect, the mouthpiece can be a single use, disposable component that can be sanitized prior to use or easily sanitizable and reusable. In all the aspects of the embodiment, the respiratory monitor may comprise one or more humidity equilibrators to equilibrate the humidity of the breath sample. In one aspect, additional filters can be incorporated into the device for filtering out microorganisms (e.g. bacteria, fungi, virus).

### Self-Expiring Badge

After tested negative for SARS-CoV-2 infections, the subjects are issued self-expiring identification badges (also called photographic badge or health badge). Thus, the systems of present invention are equipped with self-expiring badge printers. In a most preferred object of this invention is to provide a self-expiring photographic badge that expires after a predetermined time interval. Another object of this invention is to provide a Badge, the expiration of which may be visually observed. Another object of this invention is to provide a badge which is simple to use. Another object of this invention is to provide a health badge which acts to prevent unauthorized use of the badge. Another object of this invention is to distribute the badge that is fail-safe. Yet another object of this invention is to provide a Badge which may be for single-use or re-usable. Still another object of this invention is to provide the badge that is tamper resistant. Present disclosure is directed to make all the data secure by exploring various secure mechanisms. in a non-limiting example, it is contemplated to use blockchain technology.

In a preferred embodiment, the present invention provides system and methods for continuously producing a plurality of self-expiring photographic badges. The system and methods enable the subjects to create the self-expiring photographic badge after testing negative for SARS-CoV-2 infections. In one aspect, the self-expiring identification badge expires after a predetermined time interval. In another aspect, the expiration of the badge may be visually observed. In another aspect, the health badge acts to prevent unauthorized use of the badge. In yet another aspect, the badge may be for a single-use or re-usable.

In accordance with a further aspect of the invention, the system permits subjects patients retrieve information regarding future health screening and badge expiration dates. If the badge has been expired, the customer can complete the health screening and re-validate, the same badge. Thus, the system (e.g. kiosk) includes a card reader or scanner and the customer is instructed, via the display device, to Slide, scan or insert the badge Ina slot. The data read from the card uniquely identifies the customer by using a unique alpha-numeric combination of some other type that corresponds only to the one individual customer. The data can be stored on the card in a magnetic strip, barcode, or any other suitable format for storing data, Only after the identification of the person corresponding to the data on the card has been established can any personal information be accessed. Accordingly, each customers privacy, personal health screening data, or otherwise sensitive data, is not accessible or displayed at the kiosk display device until the patient's identification is made based on data read from the card.

### Exemplary Systems of the Invention

In a preferred embodiment, there is a method for controlling the spread of microorganism infections or infectious disease. The method comprising the steps of (a) identifying a group of persons desirous of controlling the spread of microorganism infections or infectious disease; (b) periodically testing said persons in said group to obtain infection status data for each person; and (c) issuing a self-expiring identification badge upon said person in said group tests negative for said microorganism infections or infectious disease. In a most preferred aspect, infection or infectious disease is caused by Severe Acute Respiratory Syndrome Coronavirus 2 (SARS-CoV-2). In one aspect, periodically testing the persons comprising: collecting a blood sample from the person in said group and testing the blood sample for SARS-CoV-2 infection and producing a result. In a related aspect, testing is performed daily, weekly, bi-weekly, or monthly for each person of the group. In one aspect, group of persons are employees of a business entity and the self-expiring identification badge is issued to the employees of the business entity. In an important aspect, self-expiring identification badge includes information on what microorganism infections or infectious disease to test for and when to test. In a related aspect, expiration of the self-expiring badge is visually observed where the self-expiring badge includes an area wherein the expiration date of the badge is written. Further, the self-expiring badge is tampering resistant. In all aspects, identifying said group of persons comprises the step of registering each person for testing. Additionally, the infection status data is stored in a confidential database for subsequent retrieval and transmission.

In a preferred embodiment, there is a method for controlling the spread of microorganism infections or infectious disease. The method comprising: (a) identifying a group of persons desirous of controlling the spread of microorganism infections or infectious disease; (b) periodically testing said persons in said group to obtain infection status data for each person; and (c) issuing a self-expiring identification badge upon said person in said group tests negative for said microorganism infections or infectious disease where said infection or infectious disease is caused by Severe Acute Respiratory Syndrome Coronavirus 2 (SARS-CoV-2). In one aspect, periodically testing the persons comprising: collecting a blood sample from the person in said group and testing the blood sample for SARS-CoV-2 infection and producing a result. In a related aspect, testing is performed daily, weekly, bi-weekly, or monthly for each person of the group. In one aspect, group of persons are employees of a business entity and the self-expiring identification badge is issued to the employees of the business entity. In an important aspect, self-expiring identification badge includes information on what microorganism infections or infectious disease to test for and when to test. In a related aspect, expiration of the self-expiring badge is visually observed where the self-expiring badge includes an area wherein the expiration date of the badge is written. Further, the self-expiring badge is tampering resistant. In all aspects, identifying said group of persons comprises the step of registering each person for testing. Additionally, the infection status data is stored in a confidential database for subsequent retrieval and transmission.

In a preferred embodiment, the present invention provides a method for controlling the spread of Severe Acute Respiratory Syndrome Coronavirus 2 (SARS-CoV-2) infection and corona virus disease. The method comprising the steps of: (a) identifying a group of persons desirous of controlling the spread of SARS-CoV-2 infection and corona virus disease; (b) collecting a blood sample from the person in said group; (c) testing the blood sample for SARS-CoV-2 infection and producing a result; and (d) issuing a self-expiring identification badge upon said person in said group tests negative for said SARS-CoV-2 infection and corona virus disease. In a related aspect, the testing is performed daily, weekly, bi-weekly, or monthly for each person of the group. In one aspect, group of persons are employees of a business entity and the self-expiring identification badge is issued to the employees of the business entity. In all aspects of the embodiment, the self-expiring badge is for a one-time use or re-usable. In all aspects, identifying said group of persons comprises the step of registering each person for testing. Additionally, the infection status data is stored in a confidential database for subsequent retrieval and transmission.

The present invention is directed to achieve the following goals:
develop a device for easy access,
help expedite the testing process throughout the country,
establish contact tracing, help subjects with last 2 weeks of touch points,
develop an app to help customers with faster processing,
manufacture devices (kiosks) at affordable cost and scale,
distribute the kiosks at various key locations throughout the country,
make the devices secure, hack proof and tamper proof,
make the data secure, may be on a blockchain technology,
private data storage and shared only with customer's physician or assigned, physician or pharmacy of positive results to schedule an appointment with the doctor for PCR test etc.,
positive results data from lab/pharmacy/doctor to synchronize with the app/device,
all the data (except private data of customers) to store into Centers for Disease Control and Prevention (CDC) or National Contact Tracing Database, and
help business to provide an infection-free environment.

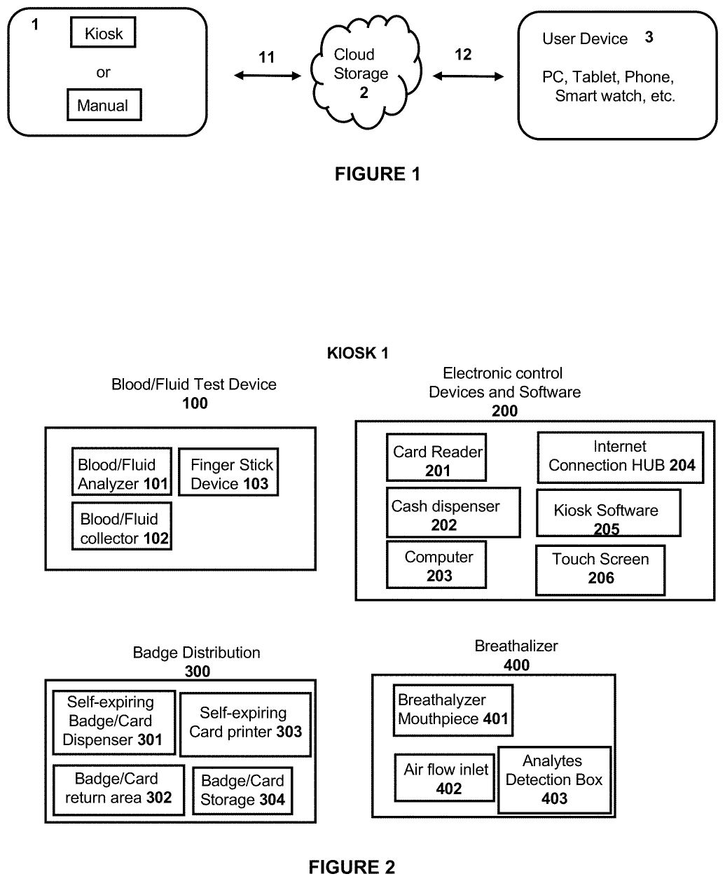

As shown in the FIG. 1, the platform of the present invention includes three sub-systems interconnected through the internet: Kiosk 1; cloud storage and infrastructure 2; and user device 3 such as a personal computer (PC), tablet computer, smart phone or smart watch. The registration system 1 and cloud storage 2 are connected by Internet connection 11 and user device 3 are connected by Internet connection 12.

The registration process can also be completed manually, with the help of a health professional or an assistant familiar with the registration process, For example, business entities can sign-up with service providers who can provide on-site registration, testing and self-expiring badge services at the business location. The employees of the business entity can complete the registration forms by physically entering all the person information and handing over the service provider and go through the subsequent testing process.

Alternatively, Kiosk can be used by any user to complete or import the registration information and collect biological samples. Kiosk is preferably placed in high traffic venues such as a retail location or a store, e.g., a pharmacy store or a supermarket, restaurants, malls, schools, airports, public places (e.g. parks), a medical office, a clinical laboratory, or a health station, or any heavy traffic moving locations that are convenient and easily accessible. The user can input the data into Kiosk via a keyboard/mouse or touchscreen, or alternatively, may input remotely from user device 3 via internet connection 12 with cloud storage 2. The data obtained by kiosk is provided to cloud storage 2 via Internet connection 11. In addition, an attendant may optionally be present at kiosk in order to assist the users operating kiosk, and to maintain and service the kiosk.

Kiosk includes a blood or fluid collector that collects blood/fluid from the kiosk user into a container. An example of such a blood/fluid collector includes, without limitation, a finger stick device that pricks the finger of the kiosk user. Kiosk retrieves the container for analysis and measurement of the collected blood/fluid. The Kiosk may also include a tissue collector (e.g. hair).

Optionally, Kiosk may also be equipped with a set of non-invasive test devices that can be used by the kiosk user to import vitals measurement data. Examples of such devices include, without limitation, weight scales, heart rate monitors/oxymeters and blood pressure devices.

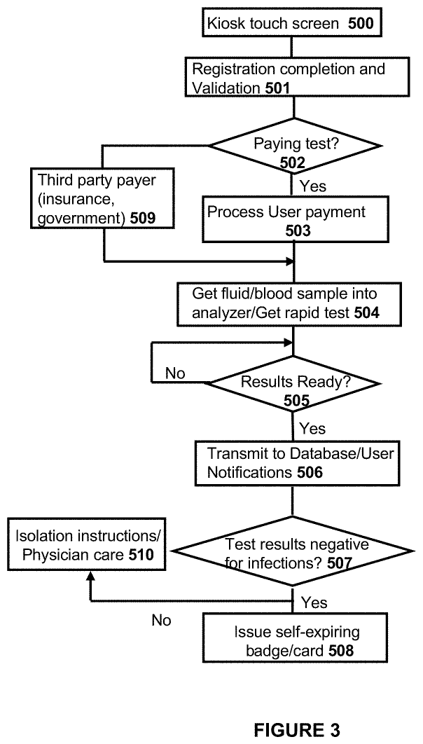

As shown in the FIG. 2, the blood/fluid test devices 100 of kiosk 1 may include, without limitation, a blood/fluid collection device 103 that draws a small amount of blood or fluid from kiosk user when the user puts the finger in the finger slot and presses the button to initiate the process; a blood/fluid collector 102 for collecting the blood/fluid collected from kiosk user; a blood/fluid analyzer 101 into which collector 102 is automatically placed. Kiosk 1 may include one or more of each of collection device 103, collector 102, and analyzer 101. When present, the optional attendant such as a qualified health processional may assist the kiosk user in using collection device 103 and placing the collected blood/fluid into collector 102 and analyzer 101. Additionally, Kiosk 1 includes a safe disposal into which contaminated medical supplies are safely disposed.

The electronic control devices and software 200 of kiosk 1 may include, without limitation, card reader 201; computer 203 including a processor; Internet connection hub 204; touch screen 206; and kiosk software 205. The kiosk user interacts with kiosk 1 via touch screen 206, with or without the help of optional attendant. The kiosk user identifies himself or herself via touch screen 206 and is guided through the process of performing selected tests by touch screen 206 and kiosk software 205. Though kiosk 1 may be used anonymously, sonic form of contact information such as an email address or mobile phone number must be provided in order for test results to be pushed electronically to the kiosk user. In addition, although a touch screen 206 is described, kiosk 1 may include alternative or additional user input mechanisms including, without limitation, keyboard, mouse or voice command input mechanisms.

If payment is required, the kiosk uses card reader 201 when prompted by touch screen 206 to scan payment information. Touch screen 206 and card reader 201 are connected to computer 203 that controls the transaction with online third-party verification software accessed through Internet connection hub 204 or through a direct Internet connection.

As shown in FIG. 2, blood/fluid analyzer 101 may be connected to hub 204 or to computer 203 using a wired connection (serial, USB or proprietary connector) or a wireless connection (WiFi, Bluetooth, ANT+ or any other wireless protocol). Hub 204 and/or computer 203 securely transmit the test measurement results from blood/fluid based test devices 100 along with personal health information obtained from the user, to cloud storage 2 or the cloud for storage and/or further user consultation. Cloud storage 2 may include databases of useful information such as payers, healthcare providers, advertisers, and health records.

Kiosk software 205 guides the interaction between kiosk 1 and the kiosk user. Kiosk software 205 implements an algorithm that is depicted by the flowchart of HG, 3. Kiosk software 205 takes the form of computer-readable program code embodied in a non-transitory computer-readable medium that is executed by a processor of kiosk computer 203 and/or cloud storage 2. In step 500, the kiosk user touches touch screen 206 in order to begin interacting with kiosk 1 or alternatively, the kiosk 1 will have motion sensors that can detect the user approaching the kiosk. In step 501, touch screen 206 displays a list of tests available to be performed by blood/fluid based test devices 100. The costs of each test may also be displayed. The kiosk user makes a selection of tests, for example, by check marking desired tests.

If a test selected by the kiosk user is a test that requires payment (step 502), then a process is initiated to verify the user and obtain payment (step 503). In particular, credit card information is obtained from the kiosk user for payment by inviting the user to swipe a credit card in card reader 201. If the insurance is paying for the test, then the user's insurance eligibility is checked and a co-pay amount, if any, is determined. If the government is paying for the test, then the user's eligibility is checked accordingly (step 509).

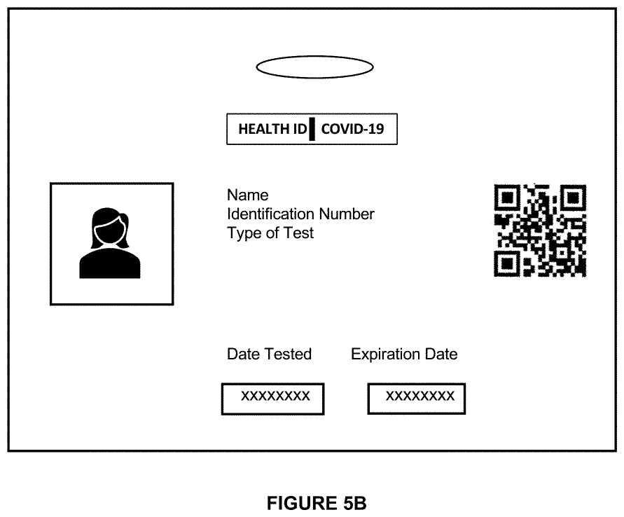

After payment is processed, the test procedure begins (step 504). If blood/fluid collection is required, the kiosk user uses collection device 103 to provide a blood fluid sample to collector 102 by a finger prick, and collector 102 is provided to analyzer 101 to begin blood/fluid analysis. Attendant, if present, can assist the kiosk user in these operations. FIG. 4 shows an exemplary rapid test cassette that can be used in the present invention. The rapid test cassette will have an identifier such as a barcode to scan the device and link to the subject.

In step 506, the test results are aggregated and transferred to cloud storage 2 or the cloud, along with an electronic notification to the user. All the data, reports, pictures and videos are stored on the secure cloud. After this, the sample is moved to safe disposal and kiosk 1 is ready for the next kiosk user.

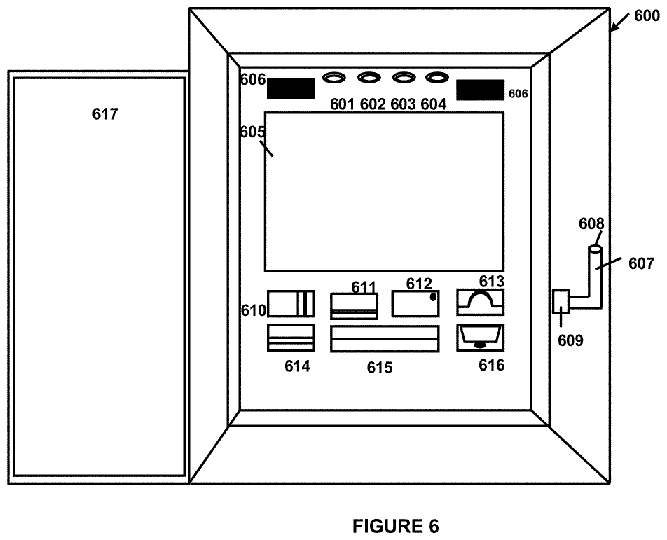

After the subjects are tested negative for selected microorganism infections (step 508), the kiosk distributes self-expiring badges to the subjects free of microorganism infections. For this, the kiosk 1 comprises a badge dispensing slot 301 through which the self-expiring badges are dispensed to subjects, a return area 302 through which the self-expiring badges are returned by subjects to the kiosk I. Also internal to the kiosk is a badge storage area 304 and a transport mechanism. The badge storage area stores badges waiting to be dispensed to a subject and receives badges that are returned by subjects. The transport mechanism is designed to transport a badge from the storage area to the slot during the issuance of a badge and transport a badge from the slot to the storage area when the badge is returned. As a result, badges that have been returned directly to the kiosk for reuse and reissue of the badges. FIG. 5 displays an exemplary self-expiring badge of the invention.

The test results along with badge details and any other relevant information may also be provided by Cloud storage 2 and/or kiosk 1 to user device 3 via Internet connection 12. A mobile application executing on user device 3 may display the test data and test result history, and may provide other options such as sharing information with a medical network or a physician; storing data in a personal health record; and making an appointment with a physician, who will be able to access the recorded health record of the consumer. The application may also display advertisements of products and services that are of relevance to the users health situation, as well as trends and progress made by the user.

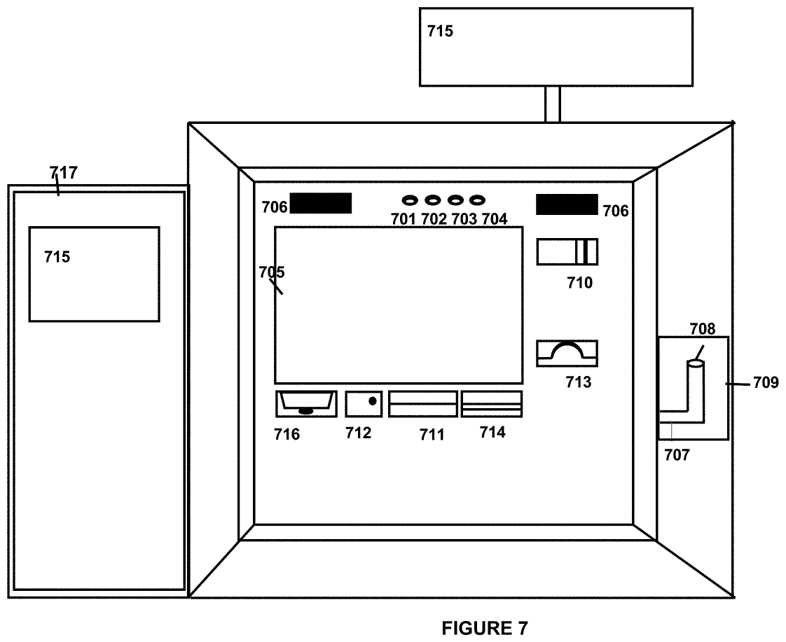

FIG. 6 and FIG. 7 shows the representative kiosks (600, 700) of the present invention. As the subject approaches the kiosk facility, the motion sensor (601, 701) detects the subject and boot up the system. The camera (60.2, 702) identifies and validates if the subject has already a registered user. A temperature sensor (603, 703) is used to measure the temperature of the kiosk user. A radar/sonar (604, 704) is used to detect the objects and the spacing. If the subjects are paying for the test, the credit card reader (610, 710) or a cash dispositor (614, 714) is used to collect the payment. If the subject orders a blood/fluid test, the subject may be provided with an alcohol swab through the disperser (615, 711) and the kiosks (600, 700) may include additional camera and a laser (not shown) to guide the user to place the finger at the right position in the finger slot (613, 713). A third camera records the rapid test process and display the process and the results on the touch screen (605, 705). FIG. 4 shows an exemplary rapid test cassette that can be used in the present invention. The kiosks can perform addition/al vital measurements as required by the tests selected by the user. For example, subject's oxygen saturation levels may be measured before the finger prick. Additionally, the dispenser (615, 711) is capable of providing respiratory masks to the users, Further, the dispenser (615, 711) can be used to provide additional items. For example, when required, the dispenser (615, 711) can provide a medical test kit comprising (a) a saliva collection kit for collecting a saliva sample; and (b) a mouth swab. In this scenario, the subject opens the kit and the mouth swab is used to swab the mouth or throat to collect a saliva sample or throat culture. The swab is placed into an envelop provided in the medical test kit and deposit (re-insert) in the dispenser. The saliva analysis may be performed at the kiosk or can be send to the off-side test lab.

The kiosks include a breathalyzer (607, 707) to determine the analytes in the subject's breath sample, The breathalyzes includes a hole (608, 708) to allow the subject to insert a tip. The tip is provided to the subject through the disperser (615, 711), The breathalyzer may be attached to a lever (609) such that the user can adjust the height of the breathalizer. Additionally, the breathalyzes can be placed in a house (709) and the subject can open the door to access the breathalyzer. All the data can be stored on the card in a magnetic strip, barcode, or any other suitable format for storing data. Kiosks are accompanied by display units (715) for advertisements of products arid services that are of relevance to the user's health situation. Alter the test results are negative for microorganism infections, the subjects are provided with self-expiring badge through the ID badge output (611) or can be the dispenser (711).

An exemplary self-expiring badge of the present invention are shown in in FIGS. 5A-5B, The badges include a picture of the customer/employee identification data such as Name, unique identification number, photo, type of test performed, data of the test and expiration date (test due date). Additionally, the badge issuance agency and the employee's business entity names can also be displayed. The unique bar code or QR code image may contain additional details about the customer/employee (e.g. test details, social security number etc.) as determined by the administrator.

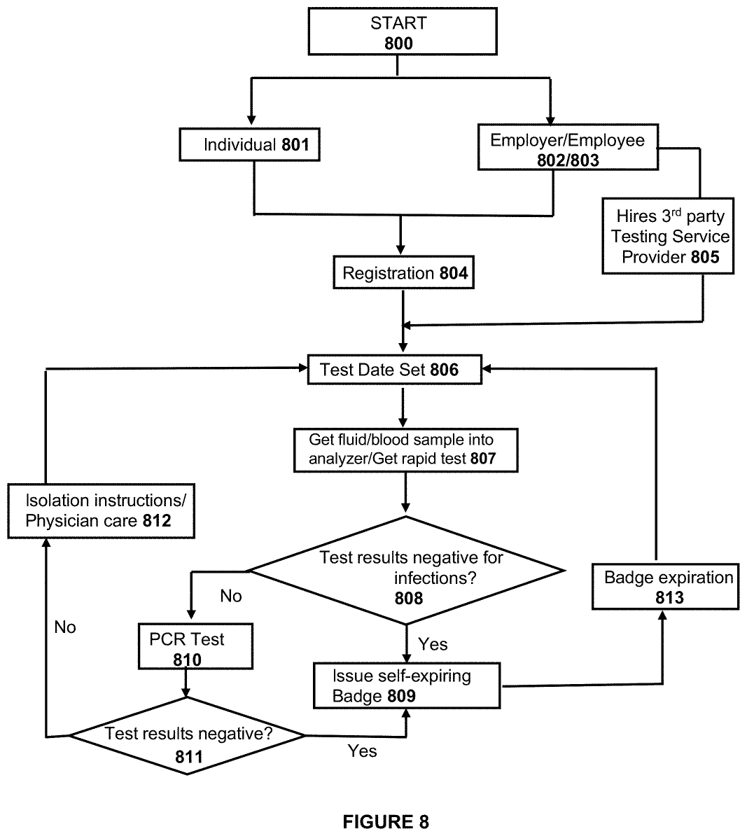

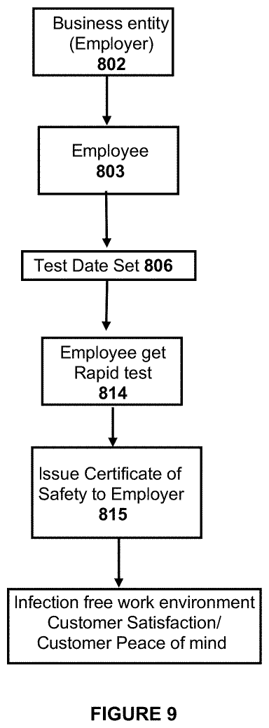

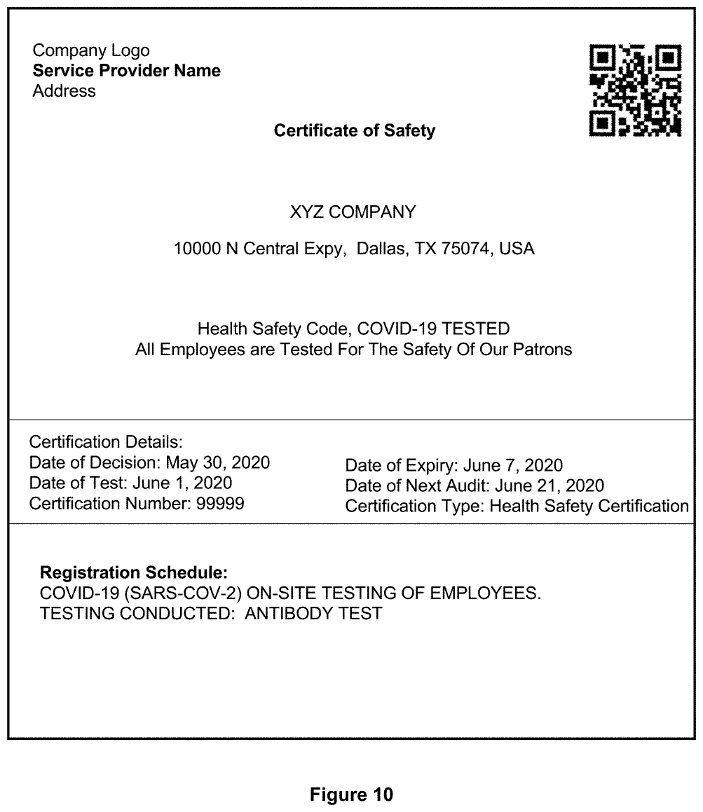

As shown in FIG. 8, individuals 801 and employers 802 can register with the service provider for health screening. The business can optionally hire a 3^{rd} party service providers to handle the administrative work, testing and badge issuance. Once the registration (step 804) is complete, the test date will be set (step 806). The employers can communicate the details about on-site testing using mass communication methods so that the employees can take advantage of the service. The individuals/employees then undergo testing and will be issued a self-expiring badge upon the test results are negative (steps 807-809), Once all the individuals/employees of the Business entity are free of infections, the Business entity may be provided with “Certificate of Safety” (step 815, FIG. 9). The Business entity may display the certificate in the visible areas of the business location so that the customers can notice and use the services and products of the business with greater confidence and peace of mind. Additionally, customers or individuals who are interested in knowing additional details can scan the OR code or barcode displayed on the “Certificate of Safety” using smart devices (phone, iPad etc.). A representational Certificate of Safety is shown in FIG. 10.

Other aspects, objects and advantages of the present invention can be obtained from a study of the drawings, the disclosure and the claims.

1. A method for controlling the spread of microorganism infections or infectious disease, said method comprising the steps of:
identifying a group of persons desirous of controlling the spread of microorganism infections or infectious disease;
periodically testing said persons in said group to obtain infection status data for each person; and
issuing a self-expiring identification badge upon said person in said group tests negative for said microorganism infections or infectious disease.

2. The method for controlling the spread of microorganism infections or infectious disease as set forth in claim 1, wherein said infection or infectious disease is caused by Severe Acute Respiratory Syndrome Coronavirus 2 (SARS-CoV-2).

3. The method for controlling the spread of microorganism infections or infectious disease as set forth in claim 1, wherein said periodically testing said persons comprising:
collecting a blood sample from the person in said group; and
testing the blood sample for SARS-CoV-2 infection and producing a result.

4. The method for controlling the spread of microorganism infections or infectious disease as set forth in claim 3, wherein said testing is performed daily, weekly, bi-weekly, or monthly for each person of said group.

5. The method for controlling the spread of microorganism infections or infectious disease as set forth in claim 1, wherein said group of persons are employees of a business entity.

6. The method for controlling the spread of microorganism infections or infectious disease as set forth in claim 5, wherein self-expiring identification badge is issued to the employees of the business entity.

7. The method for controlling the spread of microorganism infections or infectious disease as set forth in claim 1, wherein self-expiring identification badge includes information on what microorganism infections or infectious disease to test for and when to test.

8. The method for controlling the spread of microorganism infections or infectious disease as set forth in claim 1, wherein expiration of the self-expiring badge is visually observed.

9. The method for controlling the spread of microorganism infections or infectious disease as set forth in claim 1, wherein the self-expiring badge includes an area wherein the expiration date of the badge is written.

10. The method for controlling the spread of microorganism infections or infectious disease as set forth in claim 1, wherein the self-expiring badge is tampering resistant.

11. The method for controlling the spread of microorganism infections or infectious disease as set forth in claim 1, wherein the self-expiring badge is re-usable.

12. The method for controlling the spread of microorganism infections or infectious disease as set forth in claim 1, wherein said identifying said group of persons comprises the step of registering each person for testing.

13. The method for controlling the spread of microorganism infections or infectious disease as set forth in claim 1 further comprising storing infection status data in a confidential database for subsequent retrieval and transmission.

14. A method for controlling the spread of microorganism infections or infectious disease, said method comprising the steps of:
identifying a group of persons desirous of controlling the spread of microorganism infections or infectious disease;
periodically testing said persons in said group to obtain infection status data for each person; and
issuing a self-expiring identification badge upon said person in said group tests negative for said microorganism infections or infectious disease;
wherein said infection or infectious disease is caused by Severe Acute Respiratory Syndrome Coronavirus 2 (SARS-CoV-2).

15. The method for controlling the spread of microorganism infections or infectious disease as set forth in claim 14, wherein said periodically testing said persons comprising:
collecting a blood sample from the person in said group; and
testing the blood sample for SARS-CoV-2 infection and producing a result.

16. The method for controlling the spread of microorganism infections or infectious disease as set forth in claim 15, wherein said testing is performed daily, weekly, bi-weekly, or monthly for each person of said group.

17. The method for controlling the spread of microorganism infection or infectious disease as set forth in claim 14, wherein said group of persons are employees of a business entity.

18. The method for controlling the spread of microorganism infection or infectious disease as set forth in claim 14, wherein self-expiring identification badge is issued to the employees of a business entity.

19. The method for controlling the spread of microorganism infection or infectious disease as set forth in claim 14, wherein self-expiring identification badge includes information on what microorganism infection or infectious disease to test for and when to test.

20. The method for controlling the spread of microorganism infection or infectious disease as set forth in claim 14, wherein expiration of the self-expiring badge is visually observed.

21. The method for controlling the spread of microorganism infections or infectious disease as set forth in claim 14, wherein the self-expiring badge includes an area wherein the expiration date of the badge is written.

22. The method for controlling the spread of microorganism infections or infectious disease as set forth in claim 14, wherein the self-expiring badge is tampering resistant.

23. The method for controlling the spread of microorganism infections or infectious disease as set forth in claim 14, wherein the self-expiring badge is re-usable.

24. The method for controlling the spread of microorganism infections or infectious disease as set forth in claim 14, wherein said identifying said group of persons comprises the step of registering each person for testing.

25. The method for controlling the spread of microorganism infections or infectious disease as set forth in claim 14 further comprising storing infection status data in a confidential database for subsequent retrieval and transmission.

26. A method for controlling the spread of Severe Acute Respiratory Syndrome Coronavirus 2 (SARS-CoV-2) infection and corona virus disease, said method comprising the steps of:
identifying a group of persons desirous of controlling the spread of SARS-CoV-2 infection and corona virus disease;
collecting a blood sample from the person in said group;
testing the blood sample for SARS-CoV-2 infection and producing a result; and
issuing a self-expiring identification badge upon said person in said group tests negative for said SARS-CoV-2 infection and corona virus disease.

27. The method for controlling the spread of Severe Acute Respiratory Syndrome Coronavirus 2 (SARS-CoV-2) infection and corona virus disease as set forth in claim 26, wherein said testing is performed daily, weekly, bi-weekly, or monthly for each person of said group.

28. The method for controlling the spread of Severe Acute Respiratory Syndrome Coronavirus 2 (SARS-CoV-2) infection and corona virus disease as set forth in claim 26, wherein said group of persons are employees of a business entity.

29. The method for controlling the spread of Severe Acute Respiratory Syndrome Coronavirus 2 (SARS-CoV-2) infection and corona virus disease as set forth in claim 28, wherein self-expiring identification badge is issued to the employees of a business entity.

30. The method for controlling Severe Acute Respiratory Syndrome Coronavirus 2 (SARS-CoV-2) infection and corona virus disease as set forth in claim 26, wherein the self-expiring badge is re-usable.

31. The method for controlling the spread of microorganism infections or infectious disease as set forth in claim 26, wherein the self-expiring badge is for one-time use.

32. The method for controlling the spread of microorganism infections or infectious disease as set forth in claim 26, wherein said identifying said group of persons comprises the step of registering each person for testing.

33. The method for controlling the spread of microorganism infections or infectious disease as set forth in claim 26 further comprising storing infection status data in a confidential database for subsequent retrieval and transmission.
